(19) United States
(12) Patent Application Publication
(10) Pub. No.: US 2006/0194318 A1
(43) Pub. Date: Aug. 31, 2006

(54) Cultured cd14+ antigen presenting cells

(76) Inventors: Gopi Shankar, Chester Springs, PA (US); Patricia A. Lodge, Everett, WA (US); Linda A. Pestano, Oro Valley, AZ (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(21) Appl. No.: 10/544,117

(22) PCT Filed: Feb. 10, 2004
(86) PCT No.: PCT/US04/03974

§ 371 Date: Aug. 1, 2005

(30) Foreign Application Priority Data
Feb. 10, 2003 (US) 60446474

(51) Int. Cl. C12N 5/08

(52) U.S. Cl. 435/372

(57) ABSTRACT

The present invention provides isolated CD14+ antigen presenting cells, e.g., dendritic cells and isolated and enriched populations thereof as well as methods for isolation and enrichment. Also provided are methods for using the CD14+ antigenpresenting cells to modulate T cell responses in vivo, in vitro, and ex vivo.

### BACKGROUND OF THE INVENTION

Dendritic cells (DCs) play a pivotal role in immune system regulation. In addition to an important role in innate immunity, DCs provide a quantitative and qualitative framework for T cell-mediated adaptive immune responses. (See, for example, Mellman and Steinman, Cell 106:255-58 (2001); Lanzavecchia and Sallustro, Cell 106:263-266 (2001)). DCs are very effective at antigen processing and presentation, able to take up a diverse array of antigens and present them to T cells as peptides bound to both MHC class I and MHC class II molecules. Further, DCs provide other signals (generally involving, e.g., cell surface molecules and cytokines) necessary for the induction and modulation of T cell states required for an effective cell-mediated immune response. Relative to other antigen presenting cells (APCs), DCs are more adept at stimulating naïeve as well as memory T cells. Also, DCs control the quality of T cell responses by driving the differentiation of naïeve T cells into distinct classes of effectors (e.g., TH1- and TH2-differentiated cells). Thus, DCs not only generate T cells that promote the immune response, but they also can generate regulatory T cells that suppress activated T cells. (Mellman and Steinman, supra). Finally, certain DCs are believed to be capable of inducing T cell tolerance to self-antigens. (Liu, Cell 106:259-62 (2001)). Consequently, DC cellular functions are important not only for resistance to infections and tumors, but are also likely important in autoimmunity and transplant rejection.

The diverse functions of DCs in immune regulation depend in part on the diversity of DC subsets and lineages. Multiple subsets of DCs exist. (See Liu, supra). First, DCs can be classified as either immature or mature, two functionally and phenotypically distinct states. Immature DCs (imDCs) are adept at endocytosis and express relatively low levels of surface MHC class I and II and costimulatory molecules (e.g., CD80 and CD86). ImDCs, therefore, can take up antigen but generally do not present it efficiently to T cells. Recent studies suggest, however, tat, without maturation of the DCs into immunogenic form, imDCs play a toleragenic function in the immune system by presenting self-antigens to T cells. (Liu, supra; Steinman, J. Exp. Med. 191:411-416 (2000)). In this regard, it is believed that imDCs may promote naïeve CD4⁺ and CD8⁺ T cells to differentiate into IL-10 producing T regulatory/suppressor cells. (Jonuleit et al., J. Exp. Med. 192:1213-1222 (2000); Dhopadkar et al., J. Exp. Med. 193:233-238 (2001)).

ImDCs are believed to be continuously produced from hematopoietic stem cells in the bone marrow. CD34⁺ common myeloid progenitors (CMP), derived from CD34⁺ stem cells, are believed to differentiate into CD34⁺ CLA⁺ and CD34⁺ CLA⁻ populations, which subsequently differentiate into CD11c⁺ CD1a⁺ and CD11c^{+ CD}1a⁻ imDCs, respectively. (Liu, supra; Strum et al., J. Exp. Med. 185:1131-1136 (1997)). CD11c⁺ CD1a⁺ imDCs migrate into the skin epidermis to become Langerhans cells, while CD11c⁺ CD1a⁻ imDCs migrate into the skin dermis and other tissues to become interstitial imDCs. (Liu, supra; Ito et al., J. Immunol. 166:2961-2969 (2001)). The Langerhans cells and interstitial imDCs also display different functional properties. For example, interstitial imDCs, but not Langerhans cells, are able to take up large amounts of antigen by the mannose receptors and produce IL-10, possibly contributing to naïeve B cell activation and IgM production in the presence of CD40 and IL-2. (Liu, supra).

Following in vivo immunogenic challenge, e.g., by microbial infection or transplantation, imDCs undergo rapid antigen-dependent maturation into immunogenic forms. Maturing DCs rapidly lose endocytic activity, increase surface expression and stability of MHC class 1- and class II-peptide complexes, secrete proinflammatory cytokines (e.g., IL-1, IL-6, IL-12, IL-18, and IL-23), and upregulate the expression of adhesion and costimulatory surface molecules (e.g., CD40, CD54, CD80, and CD86). While mature DCs (mDCs) are less able to take up antigen relative to imDCs, these cells are extremely effective at presenting antigen and stimulating T cells (Mellman and Steinman, supra). Further, mDCs can induce different types of T cell immune responses (e.g., TH1 versus TH2) depending on the type of maturation signal (Liu, supra).

The functional diversity of different DC subsets has generated much interest in their isolation, characterization, and use in immunomodulation, both in vivo and ex vivo. (See, e.g., U.S. Pat. No. 5,994,126; U.S. Pat. No. 6,274,378; Shurin, Cancer Immunol. Immunother. 43:158-64 (1996)). Dendritic cell populations have been isolated, for example, by culturing dendritic cell precursors, obtained from peripheral blood, with various differentiation and maturation factors. (See generally, e.g., U.S. Pat. No. 6,274,378). Typically, imDCs can be obtained by culturing dendritic precursor cells in, e.g., GM-CSF and IL-4. (See for example Mellman and Steinman, Cell 106:255-58 (2001); Lanzavecchia and Sallustro, Cell 106:263-266 (2001)). Also, maturation of imDCs into mDCs can be triggered by products of microbial or viral pathogens, e.g., LPS, or by proinflammatory cytokines, e.g., TNF-α. (Mellman and Steinman, supra).

These isolated DC populations have been characterized based on, for example, expression of cell surface molecules as well as their ability to take up and present antigen. In this regard, while CD14, an LPS receptor, is abundantly expressed on a large population of peripheral blood monocytes, both imDCs and mDCs generated from monocytic DC precursors are typically characterized as lacking high CD14 expression. (See, e.g., U.S. Pat. No. 5,994,126; Czerniecki et al., J. Immunol. 159:3823-37 (1997); Sallusto and Lanzavecchia, J. Exp. Med. 179:1109-1118 (1994); Thomas et al., J. Immunol. 151:6840-6852, (1993a); Thomas et al., J. Immunol. 150:821-834 (1993b)). Thus, lack of surface CD14 has been viewed as a marker for the DC phenotype. (See, e.g., Steinman, Ann. Rev. Immunol. 9:271-296 (1991); U.S. Pat. No. 5,994,126; Czerniecki et al., supra; Sallusto and Lanzavecchia, supra; Thomas, supra (1993a); Thomas, supra (1993b)). As a result, CD14-expressing cell populations exhibiting the characteristics of antigen preventing cells, i.e., DCs, have not been appreciated nor have methods for their use in immunomodulation been developed.

Because the diverse functions of DCs depends on the multiplicity of dendritic cell subsets and lineages, the identification and isolation of specific DC subsets can provide particular cellular compositions for modulating immune responses. Hence, there is a need in the art for additional isolated DC subset populations exhibiting in vivo and ex vivo immunomodulatory capabilities.

### BRIEF SUMMARY OF THE INVENTION

The present invention provides a substantially isolated population of antigen presenting cells comprising as a component of the cell population a group of antigen presenting cells expressing the cell surface markers CD11c⁺, and CD14⁺. The CD11c⁺, CD14⁺ dendritic cells can be substantially enriched.

In one embodiment of the present invention the CD11c⁺, CD14⁺ dendritic cell population comprises a cell population substantially enriched for either immature or mature dendritic cells. The substantially enriched immature or mature dendritic cell populations can further comprise a predetermined antigen In the context of the present invention the predetermined antigen can be of any type that comprises epitopes that can be presented by dendritic cells. These antigens can include, but are not limited to, a tumor-specific antigen, a tumor-associated antigen, an autoantigen, a bacterial antigen, or a viral antigen and the like. The antigen can be provided to the dendritic cell population as a whole cell, a lysate, a membrane preparation, a partially purified preparation, a substantially purified preparation, as a recombinantly expressed protein or portion thereof, a peptide, or expressed on the surface of a recombinant cell, a liposome, or any other means.

In a particular embodiment the substantially isolated CD11c⁺, CD14⁺ dendritic cell population further comprises a tumor antigen associated with prostate cancer. Specifically, the tumor-associated antigen is prostate specific antigen (PSA), prostate specific membrane antigen (PSMA), or prostatic acid phosphatase (PAP), and the like.

In yet another embodiment of the present invention the isolated CD11c⁺, CD14⁺ dendritic cell population further comprises at least one cytokine. In particular, the cytokine is a proinflammatory or a anti-inflammatory cytokine. Specifically, the proinflammatory cytokine can be tumor necrosis factor a (TNFa), interleukin 13 (IL-13), or CD40 ligand (CD40L, also referred to as gp39). The anti-inflammatory cytokine can be interleukin 10 (IL10), tumor growth factor-β (TGF-β), or prostaglandin E₂ (PGE₂).

Another embodiment of the present invention comprises an isolated population of CD11c⁺, CD14⁻ dendritic cells and further comprising a population of T cells. Typically, the population of T cells can be any cell population comprising T cells, such as PBMCs, a cell population enriched for T cells, or a population of substantially isolated T cells. The T cells can be either autologous, syngeneic or allogeneic to the dendritic cells. In certain embodiments of the present invention the T cells population can be substantially enriched for CD4⁺ T cells, or CD8⁺ T cells.

In still another embodiment of the present invention, compositions are provided comprising an isolated population of CD11c⁺, CD14⁺ dendritic cells and further comprising a population of natural killer (NK) cells. typically, the population of NK cells can be any population of cells comprising NK cells, such as but not limited to PBMCs, a cell population enriched fro NK cells, or a population of substantially isolated NK cells. The NK cells can be autologous, syngeneic or allogeneic to the dendritic cells.

In one embodiment of the invention methods are provided for isolating a population of CD11c⁺, CD14⁺ dendritic cells comprising obtaining a population of dendritic cell precursors, differentiating the precursors into immature or mature dendritic cells, and isolating the population of CD11c⁺, CD14⁺ dendritic cells. The dendritic cell precursors can be obtained by contacting a population of leukocytes with a monocytic dendritic precursor cell-adhering substrate. Substrates useful in the present invention include,but are not limited to, glass and glass covered plastic, styrene, or polystyrene, and the like. In particular, the substrate comprises glass covered polystyrene or styrene microcarrier beads.

Differentiation of the dendritic precursor cells can be accomplished by contacting the cells with at least one cytokine. The cytokine can be granulocyte-macrophage colony stimulating factor (GM-CSF), interleukin-4 (IL-4), GM-CSF and IL-4, interleukin 13 (IL-13), or interleukin 15 (IL-15), and the like. In addition to contacting the dendritic precursor cells with a cytokine, plasma can also be included to promote the differentiation of the CD14⁺ dendritic cells.

In yet another embodiment of the present invention, the method for isolating CD11c⁺, CD14⁺ dendritic cells comprises differentiating the dendritic precursor cells or immature dendritic cells with a predetermined antigen. The predetermined antigen can comprise any antigen that can be presented by an antigen presenting cell. In a particular embodiment of the present invention the antigen is associated with prostate cancer, and can include PSMA, PSA, or PAP, and the like.

CD11c⁺, CD14⁺ dendritic cells of the present invention can be selected from a cell population comprising immature and mature dendritic cells. In one embodiment, the CD14⁺ cells are selected by admixing the population of dendritic cell precursors with a CD14 specific probe under conditions conducive to the formation of a complex with dendritic precursor cells expressing CD14, detecting the cells expressing CD14 complexed with the CD14-specific probe, and selecting the CD11c⁺, CD14⁺ dendritic cells. The CD14 specific probe can be an antibody specific for CD14, particularly a monoclonal antibody. The antibody specific for CD14 can be coupled to a solid substrate, such as a microtiter plate, a column chromatography media, or a magnetic bead, and the like. Subsequent to selecting the CD11c⁺, CD14⁺ dendritic cell precursors, the cells can be cultured under conditions conducive to the maturation of the dendritic cell precursors.

In still another embodiment of the present invention a method for modulating a T cell response to a predetermined antigen is provided. The method comprises obtaining an isolated population of CD11c⁺, CD14⁺ dendritic cells (typically immature dendritic cells or dendritic cell precursors) contacting the isolated population of CD11c⁺, CD14⁺ dendritic cells with a predetermined antigen for a time period sufficient for the dendritic cells to process the antigen, and contacting the isolated population of cells comprising CD11c⁺, CD14⁺ dendritic cells presenting processed antigen with a population of T cells to modulate the T cell response to the predetermined antigen. The CD11c⁺, CD14⁺ dendritic cells can be obtained from skin, spleen, bone marrow, thymus, lymph nodes, peripheral blood, or cord blood. The T cells can be autologous, syngeneic or allogenic to the dendritic cells and can be contacted in vitro or ex vivo.

In certain embodiments of the present invention the predetermined antigen is a tumor-specific, tumor-associated, bacterial or viral antigen. More specifically, the tumor-associated antigen can be associated with prostate cancer, and in particular embodiments of the present invention the prostate antigen can be PSMA, PSA, or PAP, and the like.

The T cells of the present invention typically are provided in a mixed population of leukocytes, such as PBMCs. But, in certain embodiments of the present invention the T cells are an isolated population of T cells substantially enriched for CD4⁺ T cells, or substantially enriched for CD8⁺ T Cells, or comprise a population of mixed CD4⁺ and CD8⁺ T cells.

### BRIEF DESCRIPTION OF THE DRAWINGS

FIG. 1 depicts a typical display of surface molecules expressed on a population of unsorted DCs and PBMC-derived monocytes. Fresh blood monocytes were isolated from leukopheresis product and cultured with 500 U/ml GM-CSF and 500 U/mi IL-4 for six days. Cells were then stained with PE- or FITC-conjugated antibodies to various cell surface markers and subjected to FACS analysis to evaluate surface molecule expression. The figure depicts the fluorescence intensity histograms for DCs derived from two different donors (solid and dashed lines) and blood monocytes (filled histogram) stained with antibodies to CD54, CD83, CD80, CD86, CD40, CD11c, CD14, and HLA-DR, DP, and DQ.

FIGS. 2A and 2B depict the level of surface molecule expression on CD14⁺ and CD14⁻ DCs in contrast with their precursors, the PBMC-derived monocytes. DCs, manufactured from these human monocytic DC precursors by culture with 500 U/ml GM-CSF and 500 U/ml IL-4, double stained with FITC-conjugated antibody specific for CD14 and with PE-conjugated antibodies to various cell surface markers and subjected to FACS analysis to evaluate surface molecule expression. FIG. 2A depicts the fluorescence intensity histograms for both CD14⁺ and CD14⁻ DCs stained with antibodies to CD54, CD86, CD11c, and CD56 (solid lines) or with isotype control antibodies (filled histograms). FIG. 2B depicts the fluorescence intensity histograms for those cells stained with antibodies to CD83, CD80, CD40, and HLA-DR, DP, and DQ.

FIGS. 3A through 3C depict examples of antigen-independent potency of various combinations of CD14⁺ and CD14⁻ DCs. FIG. 3A depicts the results of a bioassay that measures the effect on the potency of CD14⁻ DCs as CD14⁺ monocytes are added. Briefly, stimulator cells (DCs or moncytes) were plated on a 96-well culture plate and sub-optimal amounts of an anti-CD3 antibody (0.005 ng/ml) and enriched T cells were added. Cells were pulsed with ³H-thymidine, further incubated, and harvested. T cell proliferation was then determined by measuring incorporated label (delta cpm). FIG. 3B depicts the antigen independent-potency of CD14⁺ and CD14⁻ DCs. Dendritic cells were separated (sorted) into CD14⁻ DCs and CD14^{low/+} DCs. Each group of cells were then tested in the antigen-independent potency bioassay. FIG. 3C depicts the antigen independent potency of CD14⁻ DCs either alone or in combination with CD14^{low/+} DCs to assess whether a mixture of these DCs would also constitute a good population of antigen presenting cells. The potency of the various groups of APCs tested were approximately equal, indicating that an APC product containing any mixed proportions of CD14⁻ and CD14⁺ DCs was equivalent in potency to DC14⁻ DCs.

FIG. 4 depicts a comparison of the antigen-independent potency of 18 batches of antigen presenting cells. The batches were tested for the proportion of CD14⁻ and CD14⁺ DCs within the cell populations. The batches were then grouped based on the proportion of CD14⁺ DCs and then tested for potency.

FIGS. 5A through 5C depict the phenotype of DCs cultured in GM-CSF alone, GM-CSF plus IL-4 or IL-15 alone as determined by the cell surface expression of CD14, CD80 and CD1a. FIG. 5A depicts the percentage of cells expressing CD14 following culture for 5 days in GM-CSF alone. FIG. 5B depicts the percentage of cells expressing CD14 following culture for 5 days in GM-CSF with IL-4. FIG. 5C depicts the percentage of cells positive for CD80, CD1a and HLA-DR of the CD14^{low} or negative DCs as compared to those cells determined to be CD14^{high} (monocytes).

FIGS. 6A and 6B depict the quantity of IL-12 and IL-10 secreted by CD14⁺ and CD14⁻ mature and immature dendritic cells. FIG. 6A depicts the secretion of IL-12 as measured by the presence of the p70 subunit of IL-12. FIG. 6B depicts the expression of IL-10.

FIGS. 7A and 7B depict the percentage (FIG. 7A) and total number (FIG. 7B) of CD8⁺ T cells that express the Vβ17 cell surface marker indicating the presence of influenza A antigen specific cytotoxic T cells in populations of T cells contacted with CD14⁺ and CD14⁻ DCs.

### DETAILED DESCRIPTION OF THE INVENTION

Isolated CD14⁺ Dendritic Cells

The present invention provides isolated antigen presenting cells, e.g., dendritic cells (DCs) that are enriched for cells that are CD14⁺ as well as isolated populations of CD14⁺ antigen presenting cells. As used herein, the term “isolated population” means a population of cells that has been removed from its native environment “CD14⁺” means that the expression level of surface CD14 is substantially equivalent to that on PBMC-derived monocytes. Such determination can be made for example, by FACS analysis using a fluorescence-conjugated anti-CD14 antibody, where the gates for “high” staining are determined by reference to positive anti-CD14 staining on the PBMC-derived monocytes. Further, CD14^{+/high} DCs are distinguishable from a population of DCs that are CD14^{low} or CD14^{dim}, wherein “CD14^{low}” or “CD14^{dim}” refers to a low level of CD14 staining by fluorescence-conjugated anti-CD14 antibodies that is slightly positive compared to that found with an irrelevant antibody but significantly lower than that found on PBMC-derived monocytes.

The term “dendritic cell” or “DC” refers to a diverse class of morphologically similar cell types, as characterized in the art, found in a variety of lymphoid and non-lymphoid tissues that are capable of taking up and presenting antigen as MHC-bound peptides. (See, for example, Steinman, Ann. Rev. Immunol. 9:271-296 (1991)). Thus, dendritic cells are HLA-DR⁺. In addition, DCs are also generally classified in the art based on the absence of other leukocyte surface markers such as CD3 (T cells), CD19 (B cells), and CD56/57 (NK cells). (See id.) Depending on dendritic cell subtype or maturation state, DCs also express other surface markers that are recognized as being characteristic of the DC phenotype. (See, e.g., Steinman, Ann. Rev. Immunol. 9:271-296 (1991); Liu, Cell 106:259-62 (2001); Thomas et al., J. Immunol. 151:68406852, (1993a); Thomas et al., J. Immunol. 150:821-834 (1993b)). Thus, the term “dendritic cell” or “DC” accords with the use of that term in the art except that, as used herein, “dendritic cells” can also express CD14 as described above. Such cells can include, for example, DCs derived in culture from monocytic dendritic precursors as well as endogenously-derived DCs present in tissues such as, for example, peripheral blood, cord blood, skin, spleen, bone marrow, thymus, and lymph nodes.

In typical embodiments, the isolated populations of CD14⁺ dendritic cells can be enriched or substantially enriched. As used herein, the term “enriched” means that the isolated population of cells is at least 30%, at least 50%, at least 75%, or at least 90% homogeneous. The term “substantially enriched” means that the isolated population of cells is at least 60%, at least 75%, or at least 90% homogeneous.

The CD14⁺ DCs can be immature or mature. The distinguishing characteristics of mature and immature dendritic cells are described in the art. (See, e.g., Liu, supra; Mellman and Steinman, supra). Generally, for example, “immature dendritic cells” or “imDCs” have moderate CD80, CD86, and MHC expression; low or no CD83 expression; are capable of efficient uptake of antigen; and exhibit a low to moderate capacity for antigen presentation. By comparison, “mature dendritic cells” or “mDCs” have upregulated CD80, CD86, MHC, and CD83 expression; exhibit a substantially reduced capacity for antigen uptake; and exhibit a high capacity for antigen presentation. In certain embodiments, the isolated populations of CD14⁺ DCs can be substantially enriched for either mDCs or imDCs.

Methods for Isolation of CD14⁺ Dendritic Cells and for Immunomodulation

CD14⁺ DCs and cell populations substantially enriched for CD14⁺ DCs can be isolated by methods also provided by the present invention. The methods generally include obtaining a population of cells that includes DC precursors, differentiation of the DC precursors into immature or mature DCs, and can also include the isolation of CD14⁺ DCs from the population of differentiated immature or mature DCs.

DC precursor cells can be obtained by methods known in the art. Dendritic cell precursors can be isolated, for example, by density gradient separation, fluorescence activated cell sorting (FACS), immunological cell separation techniques such as panning, complement lysis, rosetting, magnetic cell separation techniques, nylon wool separation, and combinations of such methods. (See, e.g., O'Doherty et al., J. Exp. Med. 178:1067-76 (1993); Young and Steinman, J. Exp. Med. 171:1315-32 (1990); Freudenthal and Steinman, Proc. Natl. Acad. Sci. USA 87:7698-702 (1990); Macatonia et al., Immunol. 67:285-89 (1989); Markowicz and Engleman, J. Clin. Invest. 85:955-61 (1990) (each incorporated by reference herein). Methods for immuno-selecting dendritic cells include, for example, using antibodies to cell surface markers associated with dendritic cell precursors, such as anti-CD34 and/or anti-CD14 antibodies coupled to a substrate. (See, e.g., Bernhard et al., Cancer Res. 55:1099-1104, 1995; Caux et al., Nature 360:258-61, 1992 (each incorporated by reference herein).)

Enriched populations of DC precursors can also be obtained. Methods for obtaining such enriched precursor populations are known in the art. For example, enriched populations of DC precursors can be isolated from a tissue source by selective removal of cells that adhere to a substrate. (See, e.g., U.S. Pat. No. 5,994,126, incorporated by reference herein.) Using a tissue source such as, e.g., bone marrow or peripheral blood, adherent monocytes can be removed from cell preparations using a commercially-treated plastic substrate (e.g., beads or magnetic beads) to obtain a population enriched for nonadherent DC precursors. (See id.)

Monocyte DC precursors can also be obtained from a tissue source by using a DC precursor-adhering substrate. For example, peripheral blood leukocytes isolated by, e.g., leukopheresis, are contacted with a monocytic DC precursor-adhering substrate having a high surface area to volume ratio and the adherent monocytic DC precursors are separated. In additional embodiments, the substrate coupled can be a particulate or fibrous substrate having a high surface-to-volume ratio (e.g., 20 M² per liter to about 80 M² per liter), such as, for example, microbeads, microcarrier beads, pellets, granules, powder, capillary tubes, microvillous membrane, and the like. Further, the particulate or fibrous substrate can be glass, polystyrene, plastic, glass-coated polystyrene microbeads, and the like.

The DC precursors can also be cultured in vitro for differentiation and/or expansion. Methods for differentiation/expansion of DC precursors are known in the art. (See, e.g., U.S. Pat. No. 5,994,126.) Generally, expansion can be achieved by culturing the precursors in the presence of at least one cytokine that induces DC differentiation/proliferation. Typically, these cytokines are granulocyte colony stimulating factor (G-CSF) or granulocyte/macrophage colony stimulating factor (GM-CSF). In addition, other agents can be used to inhibit proliferation and/or maturation of non-DC cell types in the culture, thereby further enriching the population of DC precursors. Typically, such agents include cytokines such as, e.g., IL-13, IL-4, or IL-15, and the like. (See, e.g., id.).

Differentiation of Dendritic Cell Precursors and Promotion of the CD14⁺ Phenotype.

The isolated populations of DC precursors are cultured and differentiated to obtain immature or mature DCs. Suitable tissue culture media include, for example, but not limited to, AIM-V®, RPMI 1640, DMEM, X-VIVO 15®, and the like. The tissue culture media is typically supplemented with amino acids, vitamins, divalent cations, and cytokines to promote differentiation of the precursors toward the DC phenotype. Typically, the differentiation-promoting cytokines are GM-CSF and/or IL-4. A typical cytokine combination is about 1,000 to about 500 U/ml of GM-CSF and IL-4.

Further, cultures of DC precursors during expansion, differentiation, and maturation to the DC phenotype can include plasma to promote the development of CD14⁺ DCs. A typical plasma concentration is about 5%. In addition, where, for example, DC precursors are isolated by adherence to a substrate, plasma can be included in the culture media during the adherence step to promote the CD14⁺ phenotype early in culture. A typical plasma concentration during adherence is about 1% or more.

The monocytic dendritic cell precursors can be cultured for any suitable time. In certain embodiments, suitable culture times for the differentiation of precursors to immature dendritic cells can be about 4 to about 7 days. With the proviso that CD14 is not indicative of the lack of the DC phenotype. The differentiation of immature dendritic cells from the precursors can be monitored by methods known to those skilled in the art, such as by the presence or absence of cell surface markers (e.g., CD11c⁺, CD83^{low}, CD86^{-/low}, HLA-DR⁺ ). Immature dendritic cells can also be cultured in appropriate tissue culture medium to maintain the immature dendritic cells in a state for further differentiation or antigen uptake, processing and presentation. For example, immature dendritic cells can be maintained in the presence of GM-CSF and IL-4.

Isolation of CD14⁺ Dendritic Cells from Differentiated Dendritic Cell Precursors.

Following differentiation from DC precursors, CD14⁺ cells can be isolated to obtain an isolated population of CD14⁺ DCs. Typically, where the CD14⁺ DCs are isolated prior to maturation from enriched or substantially enriched DCs (determined by monitoring differentiation as described above), the isolated population will be enriched or substantially enriched for immature CD14⁺ DCs. Generally, isolation of the CD14⁺ DCs includes contacting the cell population from which the CD14⁺ cells are to be isolated with a CD14-specific probe. In one exemplary embodiment, CD14-expressing cells are detected by FACS using a CD14-specific probe either directly conjugated to a fluorescent molecule (e.g., FITC or PE) or with a unlabeled antibody specific for CD14 and a labeled second antibody specific for the first antibody. CD14⁺ cells can also be separated from CD14^{low} and CD14⁻ cells by FACS sorting. Gating for CD14^{high} positivity can be determined in reference to CD14 staining on, e.g., PBMC-derived monocytes. Typically, the CD14-specific binding agent is, for example, an anti-CD14 antibody (e.g., monoclonal or antigen binding fragments thereof). A number of anti-CD14 antibodies suitable for use in the present invention are well known to the skilled artisan and many can be purchased commercially.

In another embodiment, a CD14-specific probe is coupled to a substrate and the CD14⁺ cells are isolated by affinity selection. A population of cells that includes CD14⁺ cells is exposed to the coupled substrate and the CD14⁺ cells are allowed to specifically adhere. Non-adhering CD14⁻ cells are then washed from the substrate, and the adherent cells are then eluted to obtain an isolated cell population substantially enriched in CD14⁺ DCs. The CD14-specific probe can be, for example, an anti-CD14 antibody. The substrate can be, for example, commercially available tissue culture plates or beads (e.g., glass or magnetic beads). Methods for affinity isolation of cell populations using substrate-coupled antibodies specific for surface markers are generally known. (See, e.g., Bernhard et al., supra; Caux et al., supra).

Contacting Immature Dendritic Cells with Antigen and Dendritic Cell Maturation.

During culture, immature dendritic cells (either an isolated population of CD14⁻ imDCs or total imDCs prior to isolation) can optionally be exposed to a predetermined antigen. Suitable predetermined antigens can include any antigen for which T-cell modulation is desired. In one embodiment, immature dendritic cells are cultured in the presence of prostate specific membrane antigen (PSMA) for cancer immunotherapy and/or tumor growth inhibition. Other antigens can include, for example, bacterial cells, viruses, partially purified or purified bacterial or viral antigens, tumor cells, tumor specific or tumor associated antigens (e.g., tumor cell lysate, tumor cell membrane preparations, isolated antigens from tumors, fusion proteins, liposomes, and the like), recombinant cells expressing an antigen on its surface, autoantigens, and any other antigen. Any of the antigens can also be presented as a peptide or recombinantly produced protein or portion thereof. Following contact with antigen, the cells can be cultured for any suitable time to allow antigen uptake and processing, to expand the population of antigen-specific dendritic cells, and the like (see below).

For example, in one embodiment, the immature DCs can be cultured following antigen uptake to promote maturation of the imDCs into mature DCs that present antigen in the context of MHC molecules. Methods for DC maturation are known. (See, e.g., U.S. Pat. No. 6,274,378, incorporated by reference herein.) Such maturation can be performed, for example, by culture in the presence of known maturation factors, such as cytokines (e.g., TNF-α, IL-1β, or CD40 ligand), bacterial products (e.g., LPS or BCG), and the like. The maturation of imDCs to mDCs can be monitored by methods known in the art, such as, for example by measuring the presence or absence of cell surface markers (e.g., upregulation of CD83, CD86, and MHC molecules) or testing for the expression of mature dendritic cell specific mRNA or proteins using, for example, an oligonucleotide array.

Optionally, the imDCs can be cultured in an appropriate tissue culture medium to expand the cell population and/or maintain the imDCs in state for further differentiation or antigen uptake. For example, imDCs can be maintained and/or expanded in the presence of GM-CSF and IL-4. Also, the imDCs can be cultured in the presence of anti-inflammatory molecules such as, for example, anti-inflammatory cytokines (e.g., IL-10 and TGF-β) to inhibit imDC maturation.

In another aspect, the isolated population of CD14⁺ DCs are enriched for mature DCs. The isolated population of CD14⁺ mDCs can be obtained by culturing an isolated population of CD14⁺ imDCs in the presence of maturation factors as described above (e.g., bacterial products, and/or proinflammatory cytokines), thereby inducing maturation. Optionally, a mixed population of CD14⁺ and CD14⁻ imDCs (differentiated from DC precursors) can be cultured to induce maturation, the maturation stage monitored as described above, and, at the appropriate stage of mDC enrichment, the CD14⁺ cells separated as described above to obtain an isolated population enriched or substantially enriched for CD14⁺ mDCs.

According to yet another aspect of the invention, DC's can be preserved, e.g., by cryopreservation either before exposure or following exposure to a prostate cancer antigen. Cryopreservation agents which can be used include but are not limited to dimethyl sulfoxide (DMSO), glycerol, polyvinylpyrrolidone, polyethylene glycol, albumin, dextran, sucrose, ethylene glycol, i-erythritol, D-ribitol, D-mannitol, D-sorbitol, i-inositol, D-lactose, choline chloride, amino acids, methanol, acetamide, glycerol monoacetate, and inorganic salts. A controlled slow cooling rate can be critical. Different cryoprotective agents and different cell types typically have different optimal cooling rates. The heat of fusion phase where water turns to ice typically should be minimal. The cooling procedure can be carried out by use of, e.g., a programmable freezing device or a methanol bath procedure. Programmable freezing apparatuses allow determination of optimal cooling rates and facilitate standard reproducible cooling. Programmable controlled-rate freezers such as Cryomed or Planar permit tuning of the freezing regimen to the desired cooling rate curve.

After thorough freezing, DCs can be rapidly transferred to a long-term cryogenic storage vessel. In a typical embodiment, samples can be cryogenically stored in liquid nitrogen (−196° C.) or its vapor (−165° C.). Considerations and procedures for the manipulation, cryopreservation, and long term storage of hematopoietic stem cells, particularly from bone marrow or peripheral blood, is largely applicable to the DC's of the invention. Such a discussion can be found, for example, in the following references, incorporated by reference herein: Taylor et al., Cryobiology 27:269-78 (1990); Gorin, Clinics in Haematology 15:19-48 (1986); Bone-Marrow Conservation, Culture and Transplantation, Proceedings of a Panel, Moscow, Jul. 22-26, 1968, International Atomic Energy Agency, Vienna, pp. 107-186.

Frozen cells are preferably thawed quickly (e.g., in a water bath maintained at 37° -41° C.) and chilled immediately upon thawing. It may be desirable to treat the cells in 20 order to prevent cellular clumping upon thawing. To prevent clumping, various procedures can be used, including but not limited to the addition before and/or after freezing of Dnase (Spitzer et al., Cancer 45: 3075-85 (1980)), low molecular weight dextran and citrate, hydroxyethyl starch (Stiff et al., Cryobiology 20: 17-24 (1983)), and the like. The cryoprotective agent, if toxic in humans, should be removed prior to therapeutic use of the thawed DC's. One way in which to remove the cryoprotective agent is by dilution to an insignificant concentration. Once frozen DC's have been thawed and recovered, they can be used to activate T cells as described herein with respect to non-frozen DC's.

Modulation of T Cell Responses Using CD14⁺ Dendritic Cells.

According to another aspect, CD14⁺ DCs can be used to modulate T cell responses. T cells or a subset of T cells can be obtained from various lymphoid tissues for use as responder cells. Such tissues include, but are not limited to the spleen, lymph nodes, and peripheral blood. The CD14⁺ DCs can be autologous, syngeneic, or allogeneic to the T cells.

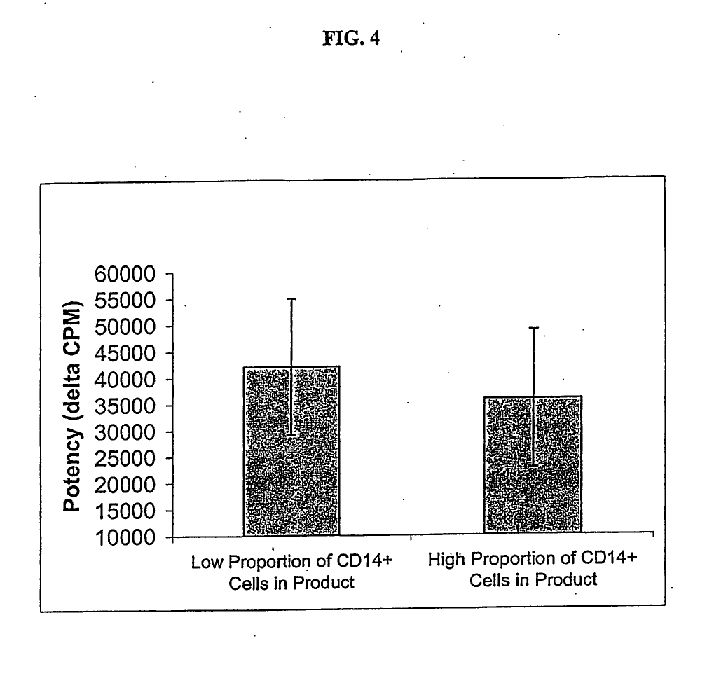

For example, CD14⁺ DCs can be used for antigen-independent T cell costimulation in vitro. (See FIG. 4, showing stimulation of T cells with CD14⁺ DCs in a DC potency/co-stimulation assay.) The T cells to be stimulated are co-cultured with an isolated population of CD14⁺ DCs. Cell activation is induced by engagement of the T cell receptor (TcR) (e.g., by contact with an anti-CD3 antibody or an antigen binding fragment thereof) or by any other agent that, at sub-optimal concentrations, provides a stimulatory signal sufficient to activate T cells in conjunction with co-stimulatory signals provided by the CD14⁺ DCs (e.g., plant lectins such as phytohemagglutinin (PHA) and the like, or mitogens of non-plant origin such as phorbol myristate acetate (PMA) and the like). Levels of T cell activation can be monitored by known methods. For example, activation can be monitored by increases in T cell proliferation (e.g., by ³H-thymidine incorporation); changes in T cell activation markers (e.g., by FACS); or changes in cytokine production (e.g., by ELISA or array).

Also, in another embodiment, CD14⁺ DCs exposed to a predetermined antigen can be used to activate T cells in vitro or ex vivo against the antigen. The CD14⁺ can be used immediately after exposure to antigen to stimulate T cells. Alternatively, DCs can be maintained in the presence of a combination of cytokines (e.g., GM-CSF and IL-4) prior to exposure to antigen and T cells. In a specific embodiment, human CD14⁺ DCs are used to stimulate human T cells.

T cells can be co-cultured with CD14⁺ DCs exposed to the predetermined antigen as a mixed T cell population or as a purified T cell subset. For example, purified CD8⁺ T cells can be co-cultured with antigen-exposed CD14⁺ DCs to elicit an antigen-specific CTL. In addition, early elimination of CD4⁺ T cells can prevent the overgrowth of CD4⁺ cells in a mixed culture of both CD8⁺ and CD4⁺ T cells. T cell purification can be achieved by positive and/or negative selection including, but not limited to, the use of antibodies directed to CD2, CD3, CD4, and/or CD8. Alternatively, mixed populations of CD4⁺ and CD8⁺ T cells can be co-cultured with CD14⁺ DCs to elicit a response specific to an antigen encompassing both a cytotoxic and T helper (T_{H}) immune response.

In addition, CD14⁺ dendritic cells contacted in vitro or ex vivo with a predetermined antigen can be used to modulate an immune response to the antigen in vivo. For example, following contact with antigen and maturation as described above, the mature, antigen-presenting CD14⁺ DCs can be administered to a human subject to stimulate an antigen-specific T cell-mediated immune response. Further, following in vitro or ex vivo activation of T cells by exposure to CD14⁺ DCs contacted with a predetermined antigen, the activated T cells can also be administered to a human subject to stimulate an immune response to the antigen.

The following examples are provided merely as illustrative of various aspects of the invention and shall not be construed to limit the invention in any way.

### EXAMPLES

### Example 1

### Promotion of CD14⁻ Dendritic Cell Phenotype Using Plasma

In the present example plasma, known to inhibit CD1a expression on dendritic cells (DCs), was tested for its ability to promote the development of the CD14⁺ phenotype when used to supplement culture medium used to culture immature DCs.

Briefly, previously frozen PBMCs and autologous plasma from a normal healthy donor were utilized. Leukopheresis material was prepared from blood obtained at two different times (herein “T1” and “T2,” approximately one year apart) from a human donor (Donor 016). The T2 leukopheresis had resulted in a large CD14⁺ DC population, while the earlier Ti leukopheresis had resulted in a “normal” (or low) percentage of the same. PBMCs from each time-point were cultured in Opti-MEM® with 5% plasma and the effects on the percentage of CD14⁺ in each cell population was analyzed. The results are shown in Table 1.

TABLE 1
CD14+ Cells (% Gated) in T1 and T2 PBMC Populations
Cultured With Either T1 or T2 Plasma

| | T2 Plasma | T1 Plasma |
|---|---|---|
| T2 PBMC | 29.36 | 14.47 |
| T1 PBMC | 1.83 | 0.47 |

These results suggested that plasma contained a factor(s) that promoted development of the CD14⁺ population; furthermore, there was a cellular component also involved such as a receptor, perhaps, for the plasma-derived factor(s). Thus, whether omission of plasma from the culture could lead to a lower percentage of CD14⁺ cells (while still supporting the generation of “good” (antigen presenting) DCs) was tested.

Since the occurrence of CD14⁺ cells after the standard DC culture procedure appeared due to a plasma component as well as a cellular component, the effects on DC culture of two other media (AIM-V® and LGM3 (also known as XVIVO-15®)) with or without 5% plasma were tested. Culture media were compared for a 6-day DC culture using Donor 016 T1 and T2 PBMCs (see above) and T2 plasma (i.e., plasma from the leukopheresis that had previously resulted in a high percentage of CD14⁺ cells). DC surface phenotype was analyzed by FACS. The results are shown in Table 2 below.

TABLE 2
Effects of Plasma Omission on DC Cultures:
DC Surface Marker Expression (% Gated)

| | CD14⁺ | CD83⁺ | CD1a⁺ | HLA-DR⁺ | CD11c⁺ |
|---|---|---|---|---|---|
| T2 Cells | | | | | |
| Opti-MEM ® | 81.23 | 10.7 | 44.07 | 89.36 | 99.29 |
| w/plasma | | | | | |
| LGM alone | 23.89 | 4.5 | 97.67 | 43.38 | 99.71 |
| LGM w/plasma | 53.18 | 14.63 | 51.16 | 85.83 | 99.35 |
| AIM-V ® alone | 3.8 | 7.79 | 78.04 | 24.6 | 99.41 |
| AIM-V ® w/plasma | 67.27 | 9.79 | 33.06 | 74.4 | 99.55 |
| T1 Cells | | | | | |
| Opti-MEM ® | 0.17 | 40.95 | 5.71 | 81.73 | 99.48 |
| w/plasma | | | | | |
| LGM alone | ˜3 | 14.46 | 84.25 | 54.76 | 99.59 |
| LGM w/plasma | 1.87 | 35.36 | 3.49 | 90.63 | 99.16 |

These data confirm that the CD14⁺ induction was related to the presence of plasma in the culture media. Further, the percentage of HLA-DR-expressing cells and CD83-expressing cells were lowered ˜50% in the absence of plasma while the percentage of CD1a⁺ cells was enhanced. Subsequent tests compared only LGM-3 (X-VIVO-15®) to the standard media (OptiMEM® plus 5% autologous plasma) since HLA-DR expression was more deficient on DC cultured in AIM-V® alone) and also because LGM-3 was known to be a good medium for DC culture.

Early Effect of Plasma on Cell Cultures:

The effect of plasma on adherence of CD14⁺ cells from the PBMC was also tested. Since a 1 hr adherence step left cells strongly adhered to the solid phase (the cells did not come off by cold PBS harvest), the DCs isolated from Patient 118 were harvested at 24 h after adherence and assayed for the presence of cell surface markers. The culture medium comprised autologous plasma Adherence of PBMCs was performed in 1% plasma followed by culture of the released DCs in 5% plasma The results are shown in Table 3 below.

TABLE 3
Early Effect of Plasma on DC Cultures:
DC Surface Marker Expression (% Total)

| Media | HLA-DR⁺ | CD3⁺ | CD19⁺ | CD14⁺ |
|---|---|---|---|---|
| Optim-MEM ® alone | 78.06 | 7.58 | 9.58 | 17.29 |
| Opti-MEM ® w/plasma | 79.45 | 10.88 | 7.71 | 44.57 |
| LGM alone | 80.05 | 6.71 | 14.12 | 13.97 |
| LGM w/plasma | 82.21 | 11.95 | 10.74 | 34.55 |
| Adhered in Opti-MEM ®; | 76.49 | 7.36 | 9.12 | 12.51 |
| cultured in LGM | | | | |

These data indicated that the effect of plasma in modulating the CD14⁺ population occurred early during culture. Such a result demonstrating the modulation of CD14 by plasma was common, but not absolute.

### Example 2

### Immunophenotyping of CD14⁺, CD14⁻, and Unsorted Dendritic Cells and PBMC-Derived Monocytes

The CD14⁺ and CD14⁻ cell populations isolated from mature DCs (obtained as described above in Example 1) were tested for expression of various cell surface molecules. Also tested were PBMC-derived monocytes that had been obtained as described above in Example 1. Cells were stained with PE- or FITC-conjugated antibodies to various cell surface markers and subjected to FACS analysis to evaluate surface molecule expression. Antibodies tested were those specific for CD54, CD83, CD80, CD86, CD40, CD11c, CD14, CD56, and HLA-DR, DP, and DQ. Matching isotypic control antibodies were also used to obtain background staining.

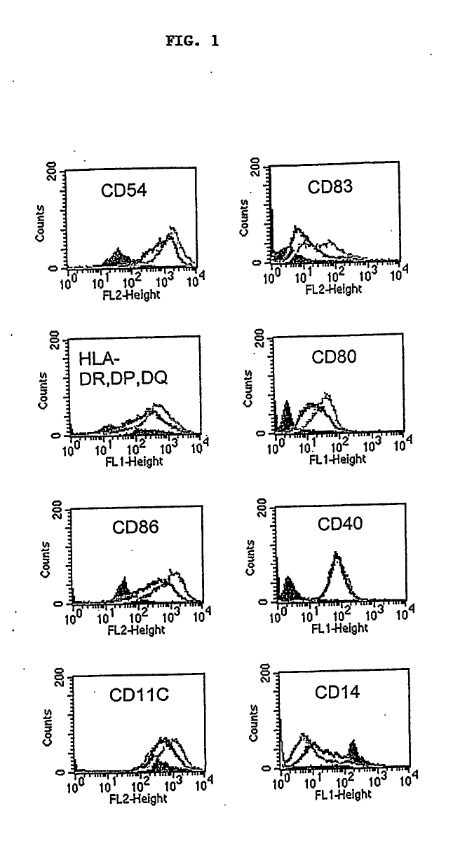

The results of immunophenotypic analysis for unsorted DCs compared to unsorted PBMC-derived monocytes are shown in FIG. 1. In this case, two lots of DCs (DCVax prostate reference cells, DCs exposed to the recombinantly expressed human PSMA (rPSMA)) were analyzed. These results demonstrated that the DCs produced were very different from blood monocytes in terms of the relative levels of expression of some cell surface molecules. Compared to blood monocytes, DCs expressed higher levels of CD54, CD80, CD83, CD86, CD40 and HLA-DR, DP, and DQ. Expression of CD11c did not differ significantly between DCs and monocytes.

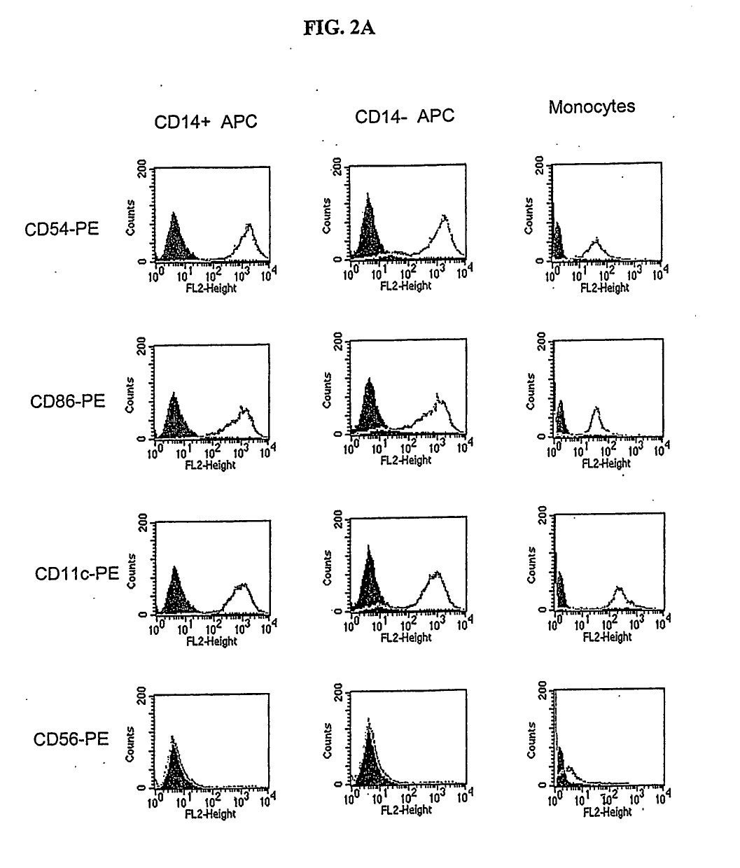

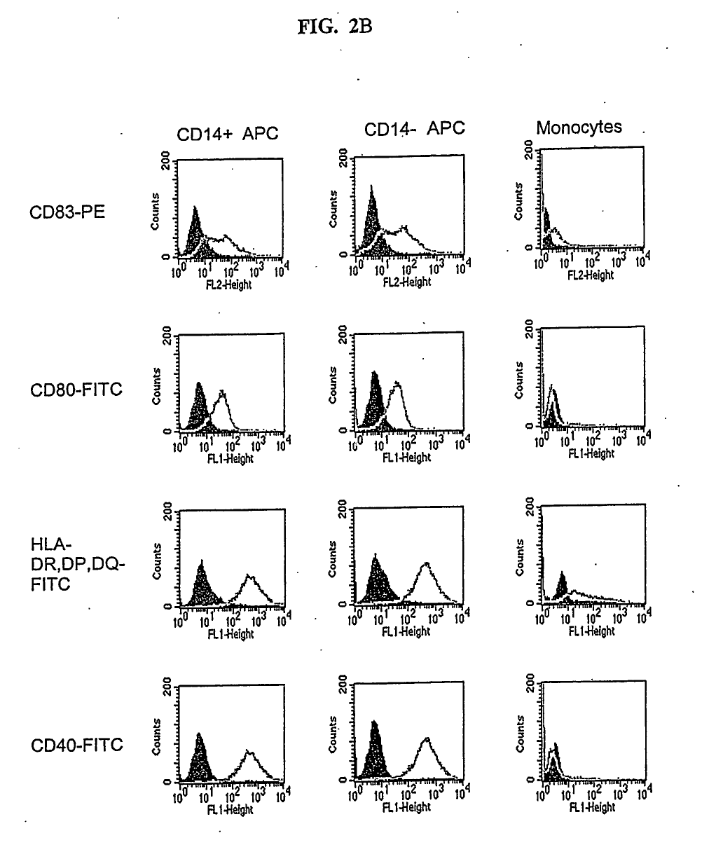

The results of immunophenotypic analysis for CD14⁺ and CD14⁻ cell populations sorted from differentiated DC precursors following contact with rPSMA and BCG are shown in FIGS. 2A and 2B. The CD14⁺ cells have identical staining patterns as CD14⁻ cells with respect to all surface molecules tested. All the markers tested were those typically expressed on mature DCs. Thus, based on immunophenotype, both the CD14⁺ and CD14⁻ cells are DCs.

The results of immunophenotypic analysis for CD14⁺ and CD14⁻ cells sorted from blood monocytes are also shown in FIGS. 2A and 2B. Comparison of marker expression on the blood monocytes with that on the DCs shown in FIGS. 2A and 2B demonstrated that the blood monocytes differed significantly from CD14⁺ DCs in terms of cell surface marker expression, exhibiting lower levels of CD54, CD86, CD80, CD40, and HLA-DR, DP, and DQ.

### Example 3

### Antigen-Independent Co-stimulation of T Cells

CD14⁺ DCs were tested for their ability to activate T cells in an antigen-independent co-stimulation (APC potency) assay.

PBMC's from human subjects were prepared by overlaying FICOLL® solution with leukopheresed blood diluted with buffered saline, spinning for 20 minutes at 2000 rpm, and isolating the white cells at the interface.

Dendritic cells preparations were made from isolated PBMCs as follows: monocytic DC precursor cells from each subject were isolated by the above procedure. DC precursors were cultured for 7 days in X-VIVO 15® supplemented with 500 U/ml or 1,000 U/ml GM-CSF and 500 U/ml IL-4. The DCs were then flow cytometrically sorted into CD14⁺ and CD14⁻ populations using a FITC-conjugated anti-CD14 antibody to detect CD14 expression.

An enriched population of T cells was prepared from PBMC by incubation with anti-HLA-DR antibody conjugated magnetic beads. Following a 30 min incubation, the cells bound to the beads were removed using a magnet The HLA-DR-depleted cells comprise a substantially enriched T cell population.

The proliferation assay was performed in two experiments as follows: 1×10⁴ CD14⁺, CD14⁻, or unsorted DCs, or PBMC-derived monocytes were added to each well of a 96-well culture plate and contacted with 0.005 ng/ml anti-CD3 antibody (BD Pharmingen, San Diego, Calif.). Then 1×10⁵ enriched T cells were added, resulting in a final volume of 0.2 ml per well. The plate was incubated for about 26 hours, and then pulsed with ³H-thymidine. The plate was further incubated for about 18 hours before harvesting and determination of incorporated label.

T cell proliferation (delta counts per minute (Δ cpm)) was measured as the difference between ³H-thymidine incorporation by T cells stimulated with a sample of the DCs or a PBMC-derived monocyte preparation in the presence of anti-CD3 antibody minus ³H-thymidine incorporation by T cells stimulated with the sample of the DC preparation alone. The mean delta cpm for each dendritic cell preparation was calculated as the mean of triplicate samples.

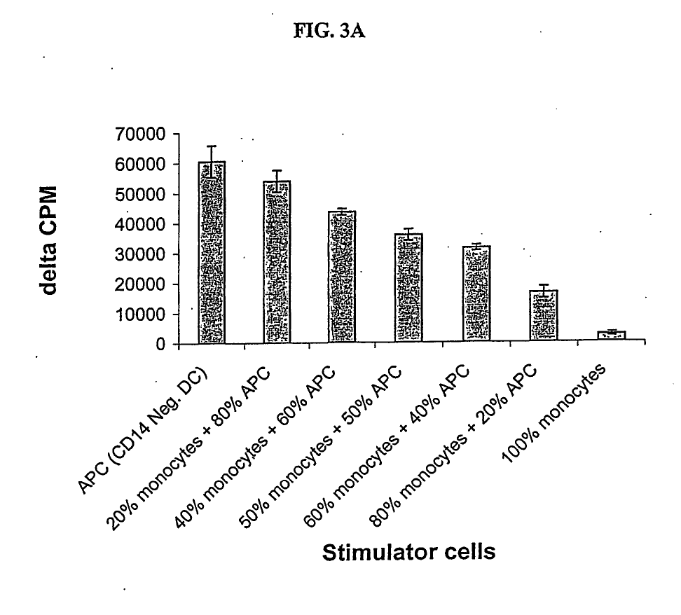

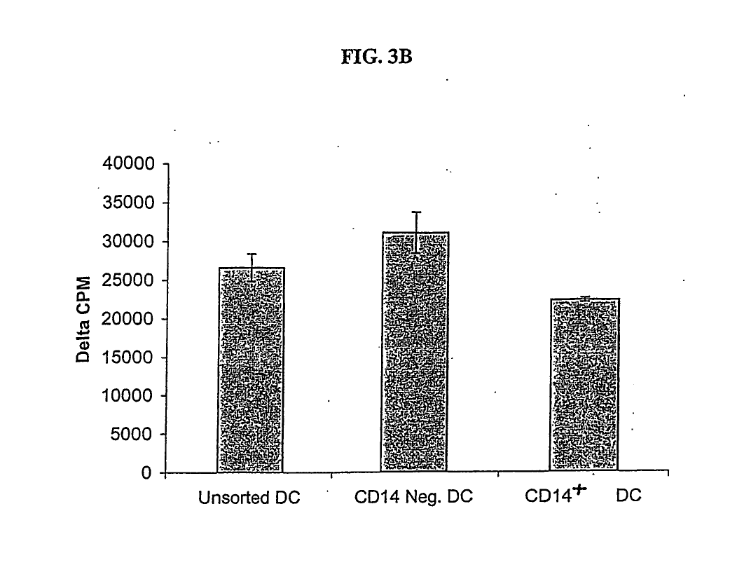

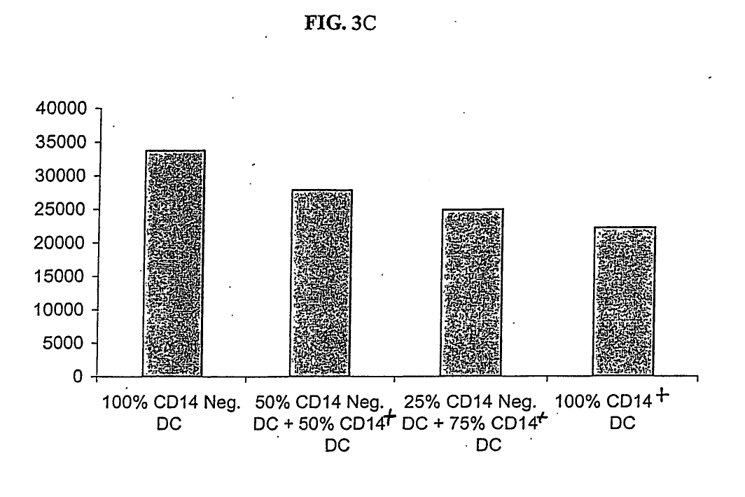

In this example, CD14⁻ APC (DCs) were found to possess an average of 60,000 Δ cpm of potency. When PBMC-derived CD14⁺ monocytes were added in increasing proportions to the APC, the potency Δ cpm values progressively declined. As indicated in FIG. 3A, the potency of monocytes alone is negligible, attesting to the fact that these cells are poor antigen presenting cells in comparison with CD14⁻ APC (DCs). In the experiment exemplified in FIG. 3B, dendritic cells were separated (sorted) into CD14⁻ DC and CD14^{low/+} DCs. The DC types were then tested in the potency bioassay. As indicated, there was no significant difference between the two groups of DCs in terms of antigen-independent potency. This proved that CD14^{low/+}-DCs, were equivalent to CD14⁻ DCs in their ability to present antigen. FIG. 3C depicts an experiment wherein CD14⁻ and CD14^{low/+} DCs, alone and in combination were tested in the potency bioassay, to assess whether a mixture of these DCs would also constitute a good population of antigen presenting cells for the activation of T cells. The potency of the various groups of APCs tested were approximately equal, indicating that an APC product containing any mixed proportions of CD14⁻ and CD14^{low/+} DCs was equivalent in potency to CD14⁻ DCs.

The above finding was confirmed by the results depicted in FIG. 4. Briefly, eighteen batches of APCs were tested for the proportion of CD14⁻ and CD14⁺ DCs. The various batches were grouped into two groups of nine samples each, based on the proportion of CD14⁺ DCs in the sample. A group was considered to contain a low proportion of CD14⁺ DCs if it had between 0.38% and 17.97% (Mean 6.36%) CD14⁺ DCs, whereas a group was considered to contain “high” proportions of CD14⁺ DCs if the group had between 20.71% and 51.90% (Mean 30.98%) CD14⁺ DCs. The potencies of the APCs in these two groups were indifferent from one another. (FIG. 4). This proved that the presence of CD14^{low/+} DCs as a mixture with CD14⁻ DCs did not lower the potency of the CD14⁻ DCs, and that mixed populations of CD14^{low/+} with CD14⁺ DCs can be used as an equivalent APC preparation for stimulating T cells.

### Example 4

### Determination of CD1a, CD80 and HLA-DR Expression

Monocytes were cultured for 5 days in XVIVO-15® plus 2% Human Serum Albumin (HSA) supplemented either with GM-CSF alone (500 U/ml), GM-CSF (500 U//ml) and IL-4 (500 U/ml), or IL-15 (100 ng/ml) alone. The resulting DCs were phenotyped for CD14 expression (FIG. 5A and 5B) as well as HLA-DR, CD80 and CD1a expression (FIG. 5C). DCs cultured in GM-CSF alone had a higher percentage of cells expressing low levels of CD14 (FIG. 5A and 5B) compared to DCs generated in GM-CSF and IL-4. However, the level of CD14 expression was much lower than that seen on monocytes cultured for the same period of time in media without GM-CSF (IL-15 only). CD14 low or negative DCs were compared to the CD14^{high} monocyte population (no GM-CSF cultures) for class II (HLA-DR), CD80 and CD1a expression. Only monocytes cultured in the presence of GM-CSF were found to express CD1a and CD80; two markers indicative of dendritic cells. (FIG. 5C) Class II expression was present on the cells from all three culture conditions.

### Example 5

### IL-10 and IL-12 Production from CD14^{low} and CD14⁻ Dendritic Cells

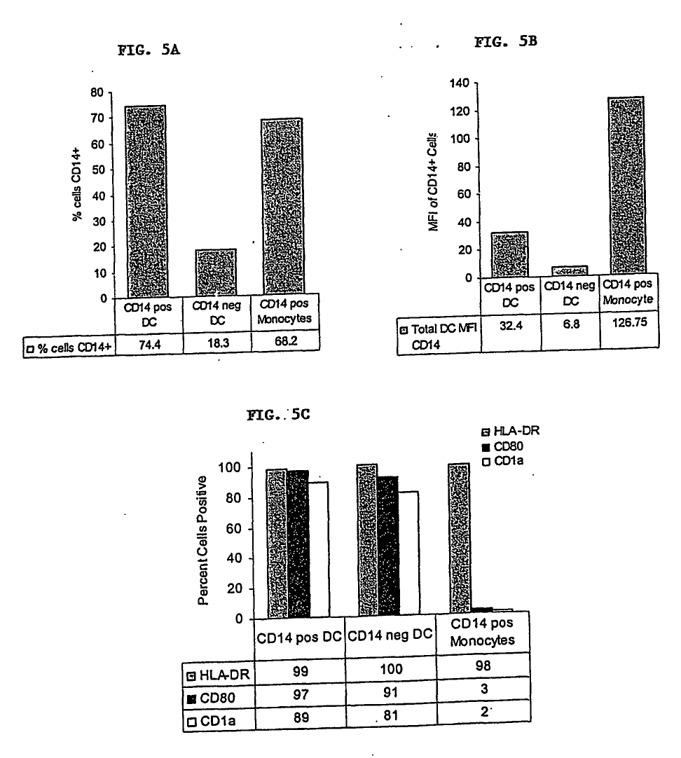

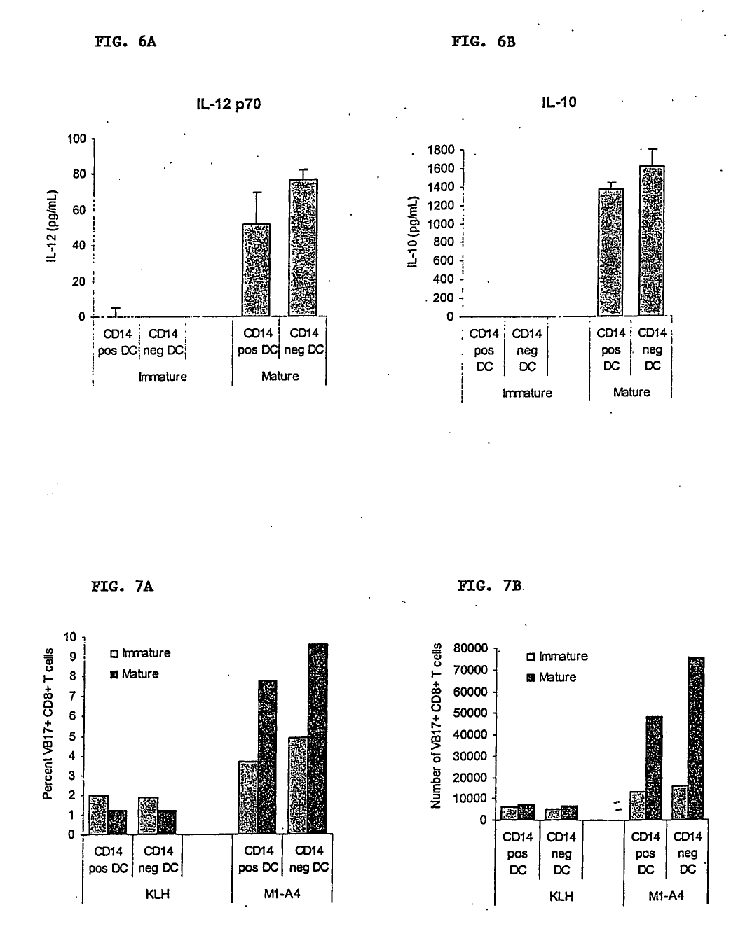

DCs generated in XVIVO-15® plus 2% HSA with either GM-CSF alone (CD14^{low}) or GM-CSF in combination with IL-4 (CD14⁻) were matured overnight with inactivated BCG (1:400 dilution) and IFN-γ (500 U/ml). Supernatants were collected from each well and run in IL-10 and IL-12p70 ELISA assays (FIG. 6A and 6B). Both DC populations produced similar amounts of IL-10 and IL-12 regardless of the level of CD14 expressed on their cell surface.

### Example 6

### Stimulation of CD8⁺ T Cell Responses

In this example, the ability of CD14^{low} and CD14⁻ DCs that were matured in the presence of BCG and IFNγ to stimulate the expansion of Vβ17⁺, CD8⁺ T cells.

The antigen-specific T cells were then co-cultured with CD14^{low} and CD14⁻ DCs. Briefly, the DCs were harvested from culture flasks and concentrated by centrifugation. For direct loading, the cells were resuspended in an equal volume of X-VIVO 15® media and influenza MI-A4 40 mer peptide containing the HLAA.A2.1 restricted epitope GlyIleLysGlyPheThrLeu (SEQ ID NO: 1) in PBS, and incubated for 1 hour at 37° C. The cells were incubated for 2 hours at 37° C. to allow for antigen processing.

DCs loaded with M1-A4 40 mer, were matured and co-cultured with autologous PBMCs (1:10 DC:PBMC ratio) for 8 days in AIM-V® plus 5% human AB sera supplemented with IL-2 (20 U/ml) and IL-15 (5 ng/ml). The resulting cells lines were analyzed for the percentage of Vβ17⁺; CD8 T cells (influenza A specific cells) in each line by flow cytometry (FIG. 7A). The absolute cell numbers were calculated by multiplying that percentage by the total cells found in each line and the results are depicted in FIG. 7B. These data demonstrated that CD14⁺ DCs as well as CD14⁻ DCs are fully capable of stimulating a substantial antigen specific CD8⁺ T cell response and that the lack of CD14 antigen on the surface of the dendritic cells is not a phenotypic characteristic linked with antigen presentation.

The previous examples are provided to illustrate, but not to limit, the scope of the claimed inventions. Other variants of the inventions will be readily apparent to those of ordinary skill in the art and encompassed by the appended claims. All publications, patents, patent applications and other references cited herein and are also incorporated by reference herein in their entirety.

What is claimed is:

1. An isolated population of antigen presenting cells expressing CD11c⁺, CD14⁺.

2. The isolated population of CD11c⁺, CD14⁺ antigen presenting cells according to claim 1, wherein the antigen presenting cells are dendritic cells.

3. The isolated cell population according to claim 2, wherein the population is enriched for the CD11c⁺, CD14⁺ dendritic cells.

4. The isolated dendritic cell population according to claim 2, wherein the dendritic cell population is substantially enriched for mature dendritic cells.

5. The isolated dendritic cell population according to claim 2, wherein the dendritic cell population is substantially enriched for immature dendritic cells.

6. The isolated dendritic cell population according to claim 2, further comprising a predetermined antigen.

7. The isolated dendritic cell population according to claim 6, wherein the predetermined antigen is a tumor-specific antigen, a tumor associated antigen, a bacterial antigen, or a viral antigen.

8. The isolated dendritic cell population according to claim 7, wherein the tumor-associated antigen is a prostate-associated antigen.

9. The isolated dendritic cell population according to claim 8, wherein the prostate-associated antigen is prostate-specific antigen (PSA), prostate-specific membrane antigen (PSMA), or prostatic acid phosphatase (PAP).

10. The isolated dendritic cell population according to claim 6, wherein the predetermined antigen is an autoantigen.

11. The isolated dendritic cell population according to claim 2, further comprising at least one cytokine.

12. The isolated dendritic cell population according to claim 11, wherein the at least one cytokine is a proinflammatory cytokine.

13. The isolated dendritic cell population according to claim 12, wherein the proinflammatory cytokine is TNFα, IL-1β, or CD40 ligand.

14. The isolated dendritic cell population according to claim 11, wherein the at least one cytokine is an anti-inflammatory cytokine.

15. The isolated dendritic cell population according to claim 14, wherein the anti-inflammatory cytokine is IL-10, TGF-β, or PGE₂.

16. The isolated dendritic cell population according to claim 2, further comprising an enriched population of T cells, or NK cells.

17. The isolated dendritic cell population according to claim 16, wherein the enriched population of T cells is a cell population comprising isolated T cells.

18. The isolated dendritic cell population according to claim 16, wherein the isolated population of T cells is substantially enriched for T cells.

19. The isolated dendritic cell population according to claim 16, wherein the dendritic cell population and the T cell population are autologous, syngeneic, or allogeneic.

20. The isolated dendritic cell population according to claim 16, wherein the T cell population is substantially enriched for CD4⁺ T cells.

21. The isolated dendritic cell population according to claim 16, wherein the T cell population is substantially enriched for CD8⁺ T cells.

22. The isolated dendritic cell population according to claim 16, wherein the T cell population is comprised of a mixed population of CD4⁺ and CD8⁺ T cells.

23. The isolated dendritic cell population according to claim 16, wherein the enriched population of NK cells is a cell population comprising isolated NK cells.

24. The isolated dendritic cell population according to claim 16, wherein the enriched population of NK cells is a cell population substantially enriched for NK cells

25. The isolated dendritic cell population according to claim 16, wherein the dendritic cell population and the NK cell population are autologous, syngeneic, or allogeneic.

26. A composition comprising an isolated population of CD11c⁺, CD14⁺ dendritic cells and a prostate-specific membrane antigen (PSMA).

27. The composition according to claim 26 ether comprising an isolated population of T cells or NK cells.

28. A method for isolating a population of CD11c⁺, CD14⁺ dendritic cells, comprising:
obtaining a population of dendritic cell precursors,
differentiating the precursors into immature or mature dendritic cells, and
selecting the population of CD11c⁺, CD14⁺ dendritic cells from the immature or mature dendritic cells.

29. The method according to claim 28, wherein the population of dendritic cell precursors is obtained by contacting a monocytic dendritic cell precursor-adhering substrate with a population of leukocytes.

30. The method according to claim 28, wherein the differentiation of dendritic cell precursors to immature and mature dendritic cells comprises culturing the precursors with at least one cytokine.

31. The method according to claim 30, wherein the at least one cytokine is GM-CSF, interleukin 4, GM-CSF and interleukin 4, interleukin 13, or interleukin 15.

32. The method according to claim 30, wherein the differentiation of dendritic cell precursors to immature and mature dendritic cells comprises culturing the precursors in the presence of plasma to promote the differentiation of the CD14⁺ dendritic cells.

33. The method according to claim 28, wherein the differentiation of dendritic cell precursors to immature and mature dendritic cells comprises culturing the precursors with a predetermined antigen.

34. The method according to claim 28, wherein the isolation of CD11c⁺, CD14⁺ dendritic cells from the immature and mature dendritic cells comprises
admixing the population of dendritic cell precursors with a CD14 specific probe under conditions conducive to the formation of a complex with the CD14 expressing dendritic cells;
detecting the CD14-expressing cells complexed with the CD14-specific probe; and
selecting the CD11c⁺, CD14⁺ dendritic cells.

35. The method according to claim 34, wherein the CD14-specific probe is a CD14-specific antibody.

36. The method according to claim 28, wherein the selection of CD11c⁺, CD14⁺ dendritic cells from the immature and mature dendritic cells comprises affinity selection of the CD14⁺ dendritic cells with a CD14-specific probe coupled to a substrate.

37. The method according to claim 36, wherein the CD14-specific probe is an anti-CD14 antibody.

38. The method according to claim 36, wherein the substrate coupled to the CD14-specific probe is a magnetic bead.

39. The method according to claim 28, further comprising culturing the CD11c⁺, CD14⁺ dendritic cells to obtain an isolated population substantially enriched for mature dendritic cells.

40. A method for modulating an T cell response to a predetermined antigen, comprising:
obtaining an isolated population of CD11c⁺, CD14⁺ dendritic cells;
contacting the isolated population of CD11c⁺, CD14⁺ dendritic cells with a predetermined antigen; and
contacting the isolated population of CD11c⁺, CD14⁺ dendritic cells with T cells to modulate the T cell response to the predetermined antigen.

41. The method according to claim 40, wherein the CD11c⁺, CD14⁺ dendritic cells have been obtained from skin, spleen, bone marrow, thymus, lymph nodes, peripheral blood, or cord blood.

42. The method according to claim 40, wherein the CD11c⁺, CD14⁺ dendritic cells and the T cells are autologous, syngeneic, or allogeneic.

43. The method according to claim 40, wherein the CD11c⁺, CD14⁺ dendritic cells are contacted with the T cells in vitro or ex vivo.

44. The method according to claim 40, wherein the predetermined antigen is a tumor-specific antigen, a tumor associated antigen, autoantigen, or a viral antigen.

45. The method according to claim 44, wherein the tumor-associated antigen is a prostate cancer-associated antigen.

46. The method according to claim 45, wherein the prostate cancer-associated antigen is prostate-specific antigen (PSA), prostate-specific membrane antigen (PSMA), or prostatic acid phosphatase (PAP).

47. The method according to claim 40, wherein the T cells are an isolated population T cells substantially enriched for CD4⁺ T cells.

48. The method according to claim 40, wherein the T cells are an isolated population of T cells substantially enriched for CD8⁺ T cells.

49. The method according to claim 40, wherein the T cells are an isolated population of T cells comprising a mixed population of CD4⁺ and CD8⁺ T cells.
